(19) United States
(12) Patent Application Publication
(10) Pub. No.: US 2017/0304039 A1
(43) Pub. Date: Oct. 26, 2017

(54) Soft Tissue Implant Systems, Kits, Implants, Devices and Associated Methods

(71) Applicants: Emory University, Atlanta, GA (US); Georgia Tech Research Corporation, Atlanta, GA (US)

(72) Inventors: Felmont Eaves, III, Atlanta, GA (US); David Ku, Atlanta, GA (US)

(21) Appl. No.: 15/492,245

(22) Filed: Apr. 20, 2017

Related U.S. Application Data
(60) Provisional application No. 62/325,174, filed on Apr. 20, 2016.

(51) Int. Cl. A61F 2/00; A61L 27/52; A61L 27/54; A61L 27/16; A61B 17/00

(52) U.S. Cl. CPC A61F 2/0059; A61L 27/16; A61B 17/00234; A61L 27/52; A61L 27/54; A61B 2017/00477; A61F 2210/0004; A61F 2210/0076; A61L 2430/34

(57) ABSTRACT

The disclosure may relate to implant systems, kits, implants, devices, and associated methods that can quickly, easily and accurately deliver a soft tissue implant with minimal incisions/trauma to surrounding tissue. The soft implant may include one or more features disposed on or within the implant configured to engage an implant insertion tool. The one or more features may include one or more of a channel disposed along the length of the soft implant, groove, slot, notch, among others, or any combination thereof. The implant insertion tool may include an implant interface section configured to engage the one or more features.

### CROSS-REFERENCE TO RELATED APPLICATIONS

This application claims the benefit of U.S. Provisional Application No. 62/325,174 filed Apr. 20, 2016. The entirety of this application is hereby incorporated by reference for all purposes.

### BACKGROUND

Soft tissue implants and fillers have been used for cosmetic or reconstructive purposes, for example, for filling wrinkles, fine lines, acne scars, contour deformities resulting from trauma, surgery, or medical therapies as well as other deformities. While implants and fillers are currently most frequently used in the face, these can be used in virtually any part of the body (e.g. hands, feet, breast, pharynx, etc.). However, the placement of implants typically requires surgical incisions that can leave visible scars and surgical development of the tissue pocket to accept implants requires surgical expertise and can be complicated by bleeding, nerve injury, or other complications. Conventional implants also can migrate, can encapsulate, or can be difficult to remove. Fillers are alternatives to facial implants include fillers but are for the short term. These also can result in bruising, can be inconsistent (e.g., lumps, irregularity, etc.), and on rare occasions can result in more significant complications, such as vascular embolism.

### SUMMARY

Thus, there is a need for an implant system that can quickly, easily and accurately position soft tissue implants through minimal incisions and with minimal dissection.

In some embodiments, the disclosure may relate to a soft tissue implant. In some embodiments, the soft tissue implant may have a length. In some embodiments, the soft tissue implant may include one or more features disposed on or within the implant configured to engage an implant insertion tool. The one or more features may include one or more of a channel, groove, slot, notch, among others, and/or any combination thereof. In some embodiments, the one or more features may include an internal channel disposed along the length of the soft tissue implant.

In some embodiments, the soft tissue implant may further include one or more regions disposed along the length, the one or more regions having one or more materials. The one or more materials may have properties configured to transform the soft tissue implant from a first configuration to a second configuration based on a stimulus. The stimulus may include thermo-manipulation, fluid manipulation, energy manipulation, chemical manipulation, and/or combination thereof.

In some embodiments, the soft tissue implant may further include one or more layers. The one or more layers may include one or more of the one or more materials, one or more therapeutics, or any combination thereof. In some embodiment, the one or more materials may include a hydrogel. In some embodiments, the hydrogel may be a polyvinyl alcohol (PVA) hydrogel.

In some embodiments, the disclosure may relate a soft tissue implant that may have a length. In some embodiments, the implant may include one or more regions disposed along the length. The one or more regions may include one or more materials. The one or more materials may have properties configured to transform from a first configuration to a second configuration based on a stimulus. In some embodiments, the stimulus may include thermo-manipulation, fluid manipulation, energy manipulation, chemical manipulation, and/or any combination thereof.

In some embodiments, the disclosure may relate to a soft tissue implant system. In some embodiments, the soft tissue implant system may include an implant insertion tool. The implant insertion tool may include an implant interface section. In some embodiments, the soft tissue implant system may include an implant including one or more features configured to engage the implant interface section.

In some embodiments, the one or more features of the implant may include one or more of a channel, a groove, a slot, a notch, among others, and/or any combination thereof. In some embodiments, the implant may include one or more regions having one or more materials. The one or more materials may have properties configured to transform the implant from a first configuration to a second configuration based on a stimulus. In some embodiments, the stimulus may include thermo-manipulation, fluid manipulation, energy manipulation, chemical manipulation, and/or any combination thereof. In some embodiments, the one or more regions may have a smooth surface, textured surface, and/or a combination thereof

In some embodiments, the implant may be disposed on the implant interface section by being fabricated directly onto the implant interface section. In some embodiments, the implant insertion tool may include a housing. The housing may include a channel disposed at least partially along a length of the housing. The channel may be in which the implant interface section is movable.

In some embodiments, the implant insertion tool may include an insert member. The insert member may be an elongated member. The insert member may include the implant interface section.

In some embodiments, the implant insertion tool may include a receiving member disposed on the housing and/or the insert member. The receiving member may be configured to support and cause release of the implant from the implant interface section. In some embodiments, the receiving member may be disposed at an end of the channel of the housing. The receiving member may be configured to support and cause the release of the implant from the interface section when the insert member is retracted into the housing.

In some embodiments, the implant interface section may include one or more engaging members configured to receive the implant. The one or more engaging members may include one or surface features. The one or more surface features may include one or more of a barb, a ball, a groove, a depression, other surface feature, and/or any combination thereof.

In some embodiments, the system may include a dissection section configured to form a soft tissue pocket or channel in a soft tissue region to receive the implant. In some embodiments, the dissection section may have a length that includes one or more surface features disposed in a pattern along the length. The one or more surface features may include one or more of a thread, a notch, a groove, a barb, a ball, other surface feature, and/or any combination thereof.

In some embodiments, the implant and/or the insertion tool may include the dissection section.

In some embodiments, the system may include a pocket development device. The pocket development device may include the dissection section.

In some embodiments, the disclosure may relate to a soft tissue implant system that includes an insertion tool. In some embodiments, the insertion tool may include an insert member, a housing, and a receiving member. In some embodiments, the insert member may be an elongated member. The insert member may include an implant interface section configured to interface with a soft tissue implant.

In some embodiments, the housing may include a channel disposed at least partially along a length of the housing. The channel may be in which the implant interface section is movable. In some embodiments, the receiving member may be disposed on the housing and/or the insert member. The receiving member may be configured to support and cause release of the implant from the implant interface section.

In some embodiments, the system may further include an implant that includes one or more features configured to engage the implant interface section. In some embodiments, the implant may include one or more regions having one or more materials. The one or more materials may have properties configured to transform the implant from a first configuration to a second configuration based on a stimulus. The stimulus may include thermo-manipulation, fluid manipulation, energy manipulation, chemical manipulation, among others, and/or combination thereof. In some embodiments, the one or more regions may have a smooth surface, a textured surface, and/or any combination thereof.

In some embodiments, the disclosure may relate to a tissue pocket development device having a length. The pocket development device may include a dissection section disposed along the length. The dissection section may be configured to form a channel or implant receiving pocket at a soft tissue incision site. The dissection section may include one or more surface features disposed in a pattern along the length. In some embodiments, the one or more surface features may include one or more of a thread, a notch, a groove, a barb, a ball, another surface feature, or a combination thereof.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIGS. 1A and 1B show examples of implants according to some embodiments;

FIG. 2 shows an example of an insertion tool according to some embodiments;

FIG. 3 shows an example of an insertion tool and an implant mounted thereon according to some embodiments;

FIGS. 4A and 4B show an example of a storage and/or casting system according to some embodiments;

FIG. 5 shows an example of an insertion tool according to some embodiments;

FIG. 6 shows an example of an insertion tool and an implant mounted thereof according to some embodiments;

FIG. 7 shows an example of a storage and/or casting system according to some embodiments;

FIG. 8 shows an example of an insertion tool according to some embodiments;

FIGS. 9A-D show an example of an insertion tool according to some embodiments;

FIG. 10 shows an insertion guide according to some embodiments;

FIGS. 11A-H show a method showing a method of inserting an implant into a soft tissue site using an insertion tool according to some embodiments;

FIG. 12 shows an example of an insertion tool according to some embodiments;

FIGS. 13A and 13B show a storage system for storing an insertion tool shown in FIG. 12 according to some embodiments;

FIGS. 14A-F show an example of an insertion tool according to some embodiments;

FIG. 15 shows an example of an insertion tool according to some embodiments;

FIGS. 16A-E show an example of an insertion tool according to some embodiments;

FIGS. 17A-E show examples of a pocket development device according to some embodiments;

FIGS. 18A and 18B show examples of a pocket development device according to some embodiments;

FIGS. 19A and 19B show an example of a pocket development device according to some embodiments; and

FIGS. 20A and 20B show an example of a pocket development device according to some embodiments; and

### DETAILED DESCRIPTION OF THE EMBODIMENTS

In the following description, numerous specific details are set forth such as examples of specific components, devices, methods, etc., in order to provide an understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosure relates to implant systems, kits, implants, devices, and associated methods that can quickly, easily, and accurately deliver a soft tissue implant with minimal incisions/trauma to surrounding tissue. In use, the implantation of the implant may require a minimal amount of or no wound closuring (e.g., limited suture placement, skin adhesives, tape closure, etc.) to close the insertion site and therefore reduce the potential for scarring. Additionally, the implant can be removed.

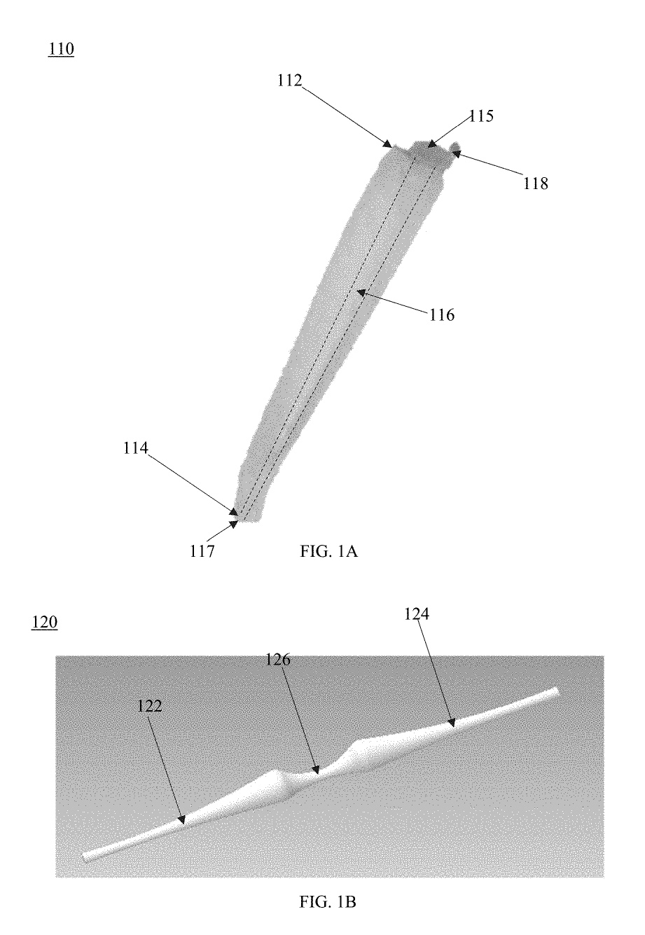

In some embodiments, the implant system may include one or more implants, implant insertion tool(s), one or more implant pocket development devices configured to provide an access channel and/or an implant receiving pocket in soft tissue at a soft tissue target site (e.g., access incision site) in which the insertion tool can be inserted to position the implant, or any combination thereof. FIGS. 1-20 show examples of implants, insertion tools, insertion guides, and pocket development devices configured to create a pocket and/or channel in the tissue in which an implant may be inserted, according to embodiments. However, it will be understood that the systems are not limited to the configuration and/or combination of the implant(s), insertion tool(s), insertion guide(s), and the pocket development device(s) as shown in and described with respect the figures. The systems may include any combination of the embodiments of, omit, and/or use alternatives for any of the pocket development device(s), insertion tool(s), insertion guide(s), and/or implant(s).

Additionally, although the implants shown in the figures may be configured to be implanted into the nasolabial fold and/or lip, the implants may be configured to fill any soft tissue area. For example, the implants may be configured to fill any facial region including but not limited to malar implant, chin implant, glabellar fold implant, nasal implant, orbital implant (e.g., infraorbital rim), or any other type of facial implant. By way of another example, the implants may be configured to fill anatomical soft tissue areas, such as defects between the metacarpal prominences of the dorsum of the hand, soft tissue depressions of the trunk or extremities secondary to steroid induced atrophy, among other areas. The systems and the disclosed devices may also be adjusted and sized for the configured implant(s), the desired point of insertion(s), among others, or any combination thereof.

In some embodiments, the implant(s) may be made of one or more materials so that it can transform the implant between a first configuration and a second configuration when exposed to a corresponding stimulus. In some embodiments, each implant may include one or more regions that are configured to change in the appearance, the function, the chemical composition, among others, or a combination thereof when interacting with a stimulus thereby changing the configuration of the implant. For example, the transformation may relate to dimensions (e.g., diameter, geometry, length, volume, etc.), firmness, composition (e.g., biodegradability, cross-linking, or other chemical changes), surface (e.g., coefficient of friction, viscosity, etc.), among others, or a combination thereof. By way of example, the one or more regions of the implant may be configured to expand and/or retract based on exposure to a stimulus to change the overall volume and thereby change the overall geometry.

In some embodiments, the one or more regions may be configured to change from the first configuration to a second configuration based on thermo-manipulation (e.g., cryo-manipulation and/or heat manipulation), fluid manipulation (e.g., dehydration and/or hydration), chemical manipulation (e.g., exposure to exogenous or endogenous chemicals or therapeutic agents), energy manipulation (e.g., delivery of ultrasonic, ultraviolet, laser, radiofrequency, etc.), among others, or a combination thereof.

For example, with respect to thermo-manipulation, the one or more regions may be configured to transform from a first configuration to a second configuration when interacting with a specific temperature, a specific range of temperatures, and/or a specific change in temperature. By way of example, the implant may be configured to be implanted in a substantially frozen state. In this example, the implant may have a first configuration in the frozen state and may be configured to transform to a second configuration when the temperature of the implant is raised to a certain temperature (e.g., body temperature when implanted). For example, the implant may have one or more regions configured to change volume and/or firmness when the temperature of the implant is raised.

For example, with respect to fluid manipulation, one or more regions may be configured to transform from a first configuration to a second configuration when interacting with specific solution(s) (e.g., water, bodily fluids, etc.), a specific amount of solution(s), among others, or a combination thereof. By way of example, the implant may be provided in dehydrated form and may change configuration when rehydrated with a solution. For example, the solution may include but is not limited to a sterile solution (e.g., saline), water, a therapeutic (e.g., lidocaine), bodily fluid (e.g., blood), among others, or a combination thereof.

In some embodiments, the implant may be made of one or more biocompatible materials. By way of example, the materials may include a hydrogel, such as a polyvinyl alcohol (PVA) hydrogel (e.g., a PVA cryo-gel), silicone, expanded polytetrafluoroethylene (ePTFE), collagen, polyethylene, other synthetic materials, among others, or a combination thereof. In some embodiments, the one or more materials may be completely or partially bio-absorbable. In some embodiments, the implant may be configured to readily removable, for example, as a single piece.

In some embodiments, the implant may include one or more therapeutic agents. By way of example, the one or more therapeutic agents may include but is not limited to anesthetic agent(s) (e.g., lidocaine), anti-inflammatory agents (e.g., steroids or NSAIDs), antibiotics, other therapeutics, among others, or a combination thereof.

In some embodiments, the implants may include one or more layers having one or more materials. By way of example, at least the outer layer may include one or more materials configured to transform when interacting with a stimulus (e.g., water, heat, cooling temperature, etc.) to thereby change the configuration of the implant. For example, the one or more regions of the implant may have the same outer layer, a different outer layer, or any combination thereof.

In some embodiments, the implant may include one or more interfacing features configured to interface with the tissue (e.g., pocket), an insertion tool and/or user before, during and/or after insertion of the implant. By way of example, the one or more interfacing features may be configured to be interface with the user directly and/or an insertion tool to provide stabilization during insertion, to assist with the positioning of the implant after insertion, to secure to the soft tissue or another implants, among others, or a combination thereof. In some embodiments, the one or more interfaces features may be disposed along the outer surface of the implant, within the implant (e.g., disposed along a surface of an internal channel), or any combination thereof. By way of example, the one or more interface features may include but are not limited to one or more tabs, barbs, other protruding members, grooves, slots, threads, channels, holes, depressions, among others, or any combination thereof.

In some embodiments, the implant may have a smooth outer surface. In some embodiments, the implant may include one or more surface features disposed along a portion of the outer surface (that contacts the soft tissue). The one or more surface features may be configured to secure the implant to the soft tissue and/or other implants. For example, the surface features may include but are not limited to groove(s), slots (s), thread(s), holes(s), other surface features, or a combination thereof. The surface features may be configured to hold the implant and/or help position when inserted into the soft tissue. By way of example, the implant may include one or more surface features (e.g., grooves, threads, etc.) that can be configured to guide the implant and/or to dissect the tissue while being inserted for implantation. In this example, the implant may act as its own pocket development device.

In some embodiments, the implant may be completely solid and/or partially solid. In some embodiments, the implant may include the one or more (internal) channels disposed along at least a portion of the length of the implant. The one or more channels may be configured to receive a portion of the insertion tool. For example, the one or more channels may extend from one end of the implant towards the opposing end. The implant may include an opening corresponding to each channel disposed at least at the one end so as to provide access to the channel. The one or more channels may be configured to receive a complimentary, corresponding portion (e.g., implant interface section) of the insertion tool. For example, the implant may include at least one channel disposed substantially in the center, offset of the center, or a combination thereof. In some embodiments, the channel may have the same diameter along the length of the channel or a different diameter (e.g., tapered diameter) along the length of the channel. In some embodiments, the dimensions of the channel (e.g., length, diameter, etc.) may correspond to the insertion tool to allow for easy insertion of the implant by a user.

In some embodiments, the implant may include one or more sections. The one or more sections may have any shape and dimensions (e.g., length, width, circumference, etc.). The shape, dimensions, or a combination thereof, of the one or more sections of the implant may depend on the anatomy, geometry, among others, or any combination thereof of the soft tissue in which the implant is configured to be implanted. For example, each section may include a concave surface, a convex surface, and/or straight surface. In some embodiments, the implant may include one section having a channel that extends from a first end towards a second, opposing end. In some embodiments, the one or more sections may include one or more regions configured to transform from a first configuration to a second configuration when exposed to a stimulus.

FIG. 1A shows an example of naso-labial fold implant 110. The implant 110 may have a first end 112, a second end 114, and a length therebetween. In this example, the implant 110 may have a tapered, cone-like shape along the length.

In some embodiments, the implant 110 may include one or more interfacing features. In the example shown in FIG. 1A, the implant 110 may include one or more open (internal) channels 116 and a tab 118. However, the implant 110 may include more, less, and/or alternative interfacing features disposed at the same and/or different locations on the implant. For example, the implant 110 may omit the one or more internal channels 116 and/or the tab 118.

In some embodiments, the one or more internal channels 116 may extend at least partially along the length of the implant 110 and the implant 110 may include at least opening corresponding to each channel disposed at one end to provide access to this channel. In this example, the (internal) channel 116 may extend along the entire length of the implant 110 from the first end 112 to the second end 114. The implant 110 may include an opening 115 disposed at the first end 112 and an opening 117 disposed at the second end 114 to correspond to the channel 116 so as to provide access to the channel 116. In other embodiments, the channel 116 may extend partially along the length from the first end 112 toward the second end 114.

In some embodiments, the tab 118 may be disposed at the first end 112. The tab 118 may be configured to be grasped by a user and/or an insertion tool to help stabilize the implant during insertion, to adjust the position of the implant after insertion, to secure the implant to the soft tissue or another implant, among others, or a combination thereof.

In some embodiments, the implant may include two or more sections. In some embodiments, the two or more sections may not be connected. For example, each section may correspond to the implant 110 shown in FIG. 1 so that the implant may include two of the implants 110 shown in FIG. 1A. By way of example, an implant having two sections that are connected, may be connected, for example, by another section. The first section and the second section may be substantially the same and/or different.

FIG. 1B shows an example of an implant 120 having two sections. The implant 120 may be configured to be implanted in the upper lip for lip augmentation. In this example, the implant 120 may include a first section 122, a second section 124 that is substantially symmetrical to a first section 122, and a third 126 section disposed between and that connects the first section 122 and the second section 124.

By way of example, an implant according to embodiments may be made by casting, molding, other fabrication methods, or a combination thereof. In some embodiments, the implant may be casted, molded, or otherwise fabricated directly onto a portion of the insertion tool using a pre-made mold specific to the device and/or implant. In other embodiments, the implant may be casted, molded, or other fabricated methods, or a combination thereof, by itself, for example, by using a mold specific to the implant and therefore can be provided separately from the insertion tool.

In some embodiments, the insertion tool may include an insert member. The insert member may include an elongated member. In some embodiments, the insertion tool may also include a housing in which and/or with respect to the insert member may be configured to move. In some embodiments, the insert member may have a length that is longer, substantially the same as, or shorter than the housing. By way of example, the housing may include one or more members that include and/or define an inner channel in which the insert member may move (e.g., retract/advance) within and with respect to. In some embodiments, the insert member may be fixed to the housing. In some embodiments, the insertion tool may omit the housing. Also, in some embodiments, the insertion tool may also act as a casting/molding mandrel.

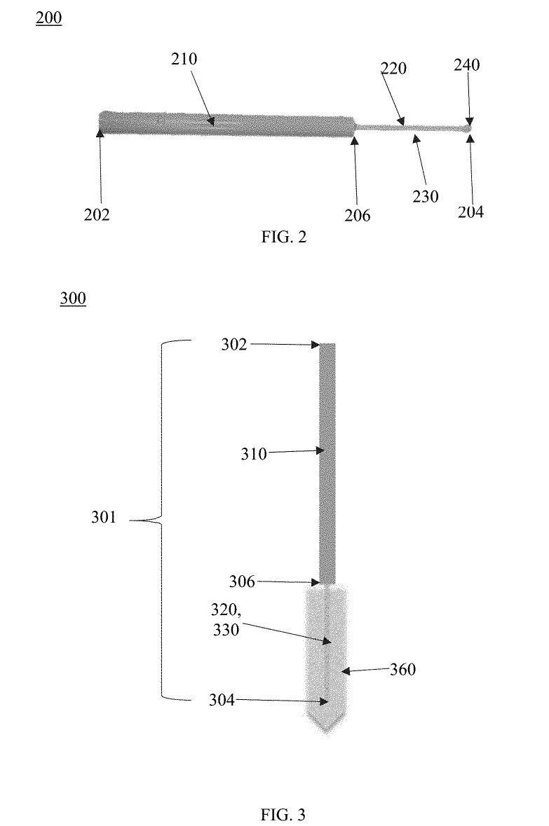

FIGS. 2-16 show examples of insertion tools according to embodiments. It will be understood that the configuration of the insertion tools are not limited to those shown and described with respect to the figures. The insertion tools may include any combination of insert member(s) and/or housing(s) according to embodiments.

In some embodiments, the insert member and/or the housing may include an implant interface section on which and/or in which an implant can be casted directly thereon/therein and/or positioned by a user. The implant interface section may be disposed along at least a portion of the length of the insert member and/or housing.

In some embodiments, the implant interface section may include one or more engaging members disposed along the length to engage the implant, for example, to improve adhesion and/or support during use and/or transport. The engaging members may be configured to engage with the implant, for example, to improve the adhesion of the implant to the implant interface section and/or act as a support for the implant. In some embodiments, the one or more engaging members may include but are not limited to barbs, balls, grooves, depressions, other textured surfaces, or any combination thereof. In some embodiments, the one or more engaging members may be retractable. For example, the one or more engaging members may be configured to retract within the implant interface section.

FIG. 2 shows an example of an insertion tool 200 according to some embodiments. In this example, the insertion tool 200 may also act as a casting/molding mandrel. Also, in this example, the insertion tool 200 may not include a housing. In some embodiments, the insertion tool 200 may include a first end 202 and a second end 204 and a length there between. In some embodiments, the insertion tool 200 may include a handle 210 and an insert member 220 disposed along the length. The handle 210 may be disposed along the length and extend between the first end 202 and position 206. In some embodiments, the handle 210 may have any diameter, shape, and/or configuration. In this example, the handle 210 may have a circular elongated shape. In other embodiments, the handle 210 may have a different shape. For example, the handle 210 may have a circular shape.

In some embodiments, insert member 220 may be disposed directly adjacent to the handle 210 and extend between the second end 204 and the position 206. In some embodiments, the handle 210 may have a different diameter and/or shape than the insert member 220. As shown in FIG. 2, the insert member 220 may include an implant interface section 230. In this example, the entire length of the insert member 220 (e.g., from the second end 204 to the position 6) may correspond to the implant interface section 230.

In some embodiments, the implant interface section 230 may be a solid elongated member. In other embodiments, the implant interface section 230 may include an inner channel. In some embodiments, the implant interface section 230 may have the same diameter and/or tapered diameter along its length. In some embodiments, the implant interface section 230 may include one or more engaging members disposed along the length.

As shown in FIG. 2, the insertion tool 200 may include an engaging member 240 disposed at the end 204. In this example, the engaging member 240 may be a circular member that protrudes from and radially extends about (e.g., around) the end 204.

FIG. 3 shows an example of a configured device 300 that includes an implant 330 and an insertion tool 301 on which the implant 330 is disposed. In this example, the insertion tool 301 may also act as a casting/molding mandrel. The insertion tool 301 may include a first end 302, a second end 304, and a length there between. Like the insertion tool 200 shown in FIG. 2, the insertion tool 301 may include a handle 310 and an insert member 320 that is disposed directly adjacent to the handle section and includes an implant interface section 330. The handle 310 may extend between the end 302 and point 306 and the insert member 320/the implant interface section 330 may extend between the end 304 and the point 306. Although not shown, the device 301 may include one or more engaging members disposed along the length of the implant interface section 330.

In some embodiments, the implant 330 may be disposed along the entire length of the implant interface section 330 and extend past the end 304. In other embodiments, the implant 360 may be disposed on the implant interface section 330 so that it extends only substantially along the length of the implant interface section 330.

In some embodiments, the implant 360 may be casted, molded, or otherwise fabricated directly onto the implant interface section 330 and/or the implant 360 may be positioned onto the implant interface section 330 by the user, during assembly or in the course of manufacturing or packaging.

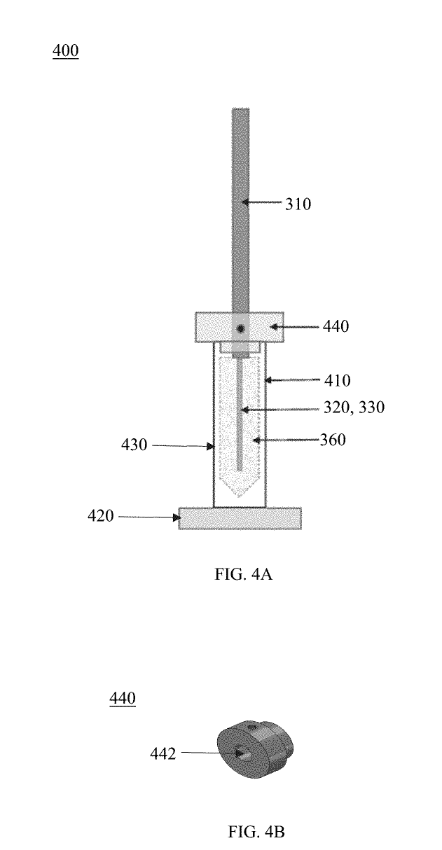

FIGS. 4A and B show an example 400 of a system 410 that can be used to cast the implant 360 onto the insertion tool 301 and/or that can be used to store the configured device 300. The system 410 may be configured to store the configured device 300 in a vertical and/or horizontal position. In some embodiments, the system 410 may include a base section 420 and a casting and/or storage section 430 disposed above the base section 420. The section 430 may be configured to cast and/or support the implant 360 when it is disposed onto the implant interface section 320. In some embodiments, the system 410 may include a cap 440 to position and support the insertion tool 301 during the curing of the implant 360 and/or storage of the configured device 300.

FIG. 4B shows an example of the cap 440. The cap 440 may include an inner opening 442 in which the handle 310 may be positioned. In some embodiments, the cap 440 may include one or more fastening members, such as pin holes, clasps, threads, or other fasteners, that can be configured to secure the cap 440 to the base 410. By way of another example, the cap 440 may be configured to snap-fit onto the base 410. In some embodiments, the base 410 may also include complimentary fastening member(s) (not shown). These fastening member(s) can be configured to release the cap 440 from the base 410 so that the cap 410 can be removed in a controlled manner during manufacturing, packaging, or by the user in the course of insertion. In some embodiments, the cap 440 may be configured to act as a receiving member (discussed in more detail below).

In some embodiments, the insertion tool may include a housing in which the insert member may move. In some embodiments, the housing may include and/or define an internal channel along its length in which and with respect to the insert member may move. In some embodiments, in which the insert member includes an implant interface section, the implant interface section may be configured to advance in a direction parallel to the housing, perpendicular to the housing, at an angle to the housing, among others, or any combination thereof

In some embodiments, the housing and/or the insert member may include one or more receiving members disposed at one end configured to support the implant, for example, during insertion into the soft tissue area by preventing the movement of the implant when the tool is being removed.

In some embodiments, the housing may include a handle disposed at the other end. In other embodiments, the handle and/or receiving member may be omitted.

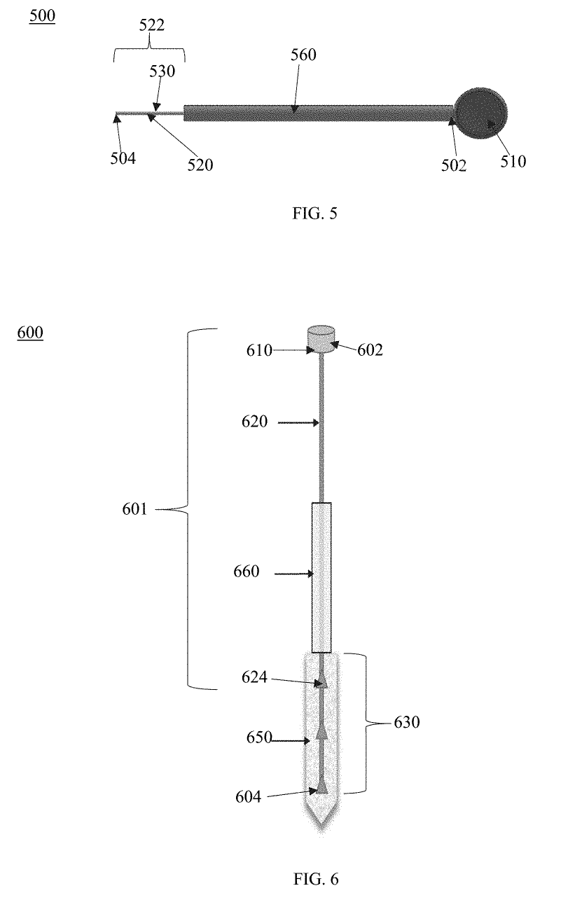

FIG. 5 show an example of an insertion tool 500 according to embodiments. As shown in FIG. 5, the tool 500 may include an insert member 520 and a housing 560 in which the insert member 520 may move. The insert member 520 may have a first end 502, a second end 504, and a length there between. In this example, the insert member 530 may be an elongated member (e.g., hollow or a solid lumen). In some embodiments, the tool 500 may include an implant interface section 530 disposed along a portion of the length of the insert member 520 (e.g., between the handle (the first end 502) and the second end 504).

In some embodiments, the insertion tool 500 may include a handle 510 disposed near the first end 502. In this example, the handle 510 may have a circular shape.

In some embodiments, the housing 550 may include an inner channel that extends along the length of the device in which the insert member 520 may move (e.g., advance/retract). In some embodiments, the housing 560 may be disposed on the insert member 520 between the handle 510 and the implant interface section 530. In some embodiments, the housing 550 may have a length that is shorter than the insert member 520. In some embodiments, the implant interface section 530 may correspond to a portion 522 of the insert member 520 near the second end 504 that is exposed when the insert member 520 is advanced through the housing 560.

FIG. 6 shows an example of a configured device 600 that includes an implant 650 and an insertion tool 601 on which the implant 650 is disposed. The insertion tool 601 may include a first end 602, a second end 604, and a length there between. Like the insertion tool 500 shown in FIG. 5, the insertion tool 601 may include a handle 610 disposed adjacent to the end 602, an insert member 620 that extends between the first end 602 (i.e., the handle 610) and the second end 604, and a housing 660 in which the insert member 620 may move.

In some embodiments, the insertion tool 601 may include an implant interface section 630 disposed along a portion of the insert member 620 adjacent to the end 604 that is exposed when the insertion member 620 is inserted in the housing 660. In some embodiments, the insertion tool 601 may include one or more engaging members 624 disposed along the length of the implant interface section 630. In this example, the one or more engaging members 624 may include a plurality of barbs. In some embodiments, the implant interface section 630 may have a length so that the implant 650 may be disposed so that the implant 650 extends over the engaging members 624 and extend past the end 604 when disposed on the implant interface section 620. In other embodiments, the implant interface section 630 may have a length so that that the implant 650 may extend only substantially along the length of the implant interface section 630 and not extend past the end 604. In some embodiments, the implant 650 may be disposed on the implant interface section 630 by being casted, molded, or otherwise fabricated directly onto the implant interface section 630 and/or by being positioned onto implant interface section 630 by the user or during the course of manufacturing or assembly.

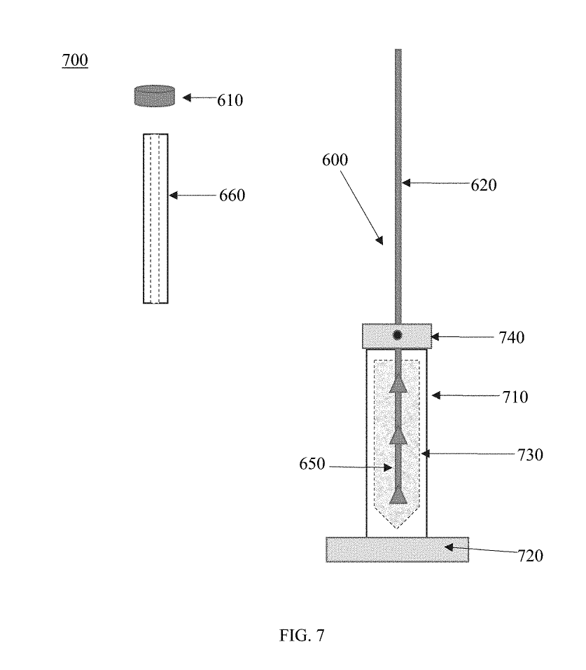

FIG. 7 shows an example 700 of a system 710 that can be used to fabricate, for example mold or cast, the implant 650 onto the insertion tool 601 resulting in the configured device 600 and/or that can be used to store the configured device 600. The fabrication/storage system 710 may be configured to store the configured device 600 in a vertical and/or horizontal position. In some embodiments, the system 710 may include a base section 720 and a casting and/or storage section 730 disposed above the base section 720. The section 730 may be configured to cast and/or support the implant 650 when it is disposed onto the implant interface section 630. By way of example, the section 730 may be a casting mold into which the insert member 620 may be positioned.

In some embodiments, the system 710 may include a cap 740 to position and support the configured device 600 during the curing of the implant 650 and/or storage. In some embodiments, the cap 740 may include channels, vents, or other features (not shown) through which casting or molding materials for the implant 650 may be inserted into the casting and/or storage section 730 or out of which air or other materials within the casting and/or storage section 730 may be removed (e.g., vented). In some embodiments, the handle 610 and the housing 660 may be provided separately from the system 710 and can be added to the configured device 600 by a user.

In some embodiments, the insertion tool may include one or more receiving members disposed at the end of the housing and/or the insert member configured to stabilize the implant during transport, loading of the implant onto the insertion tool, and/or insertion. By way of example, the one or more receiving members may be configured to provide resistance when retracting the insert member through the housing. This resistance can be used to stabilize the implant during positioning within the soft tissue space and to cause the release of the implant from the insert member/implant interface section when in use. In this way, the one or more receiving members can act as a backstop. In some embodiments, the one or more receiving members may be disposed adjacent to the end of the housing. In some embodiments, the one or more receiving members may be disposed so as to surround the inner channel of the housing. The one or more receiving members may have any shape. In some embodiments, the one or more receiving members may be disposed at other positions along the housing and/or insert member. For example, the one or more receiving members may be disposed along the length of the housing. For example, the shape of the one or more receiving members may have a shape that corresponds to the implant so to provide adequate support. In other embodiments, a cap of the fabrication/storage system (e.g., the cap 740) may be configured so as to function also as a receiving member.

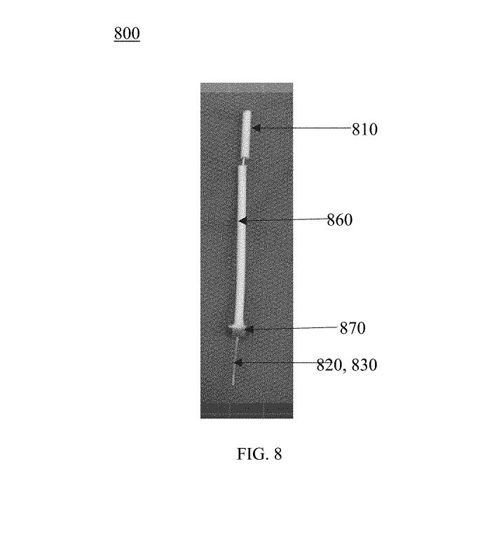

FIG. 8 shows an example of an insertion tool 800 that includes an insert member 830 and a housing 850 in which the insert member 820 may move (e.g., retract/advance). The insert tool 800 may include a handle 810 disposed one end of the insert member 820 and an implant interface section 830 disposed toward the other end of the insert member 820. In some embodiments, the insertion tool 800 may include a housing 860 that includes a receiving member 870 disposed at an end opposing the end that can interface with the handle 810 and configured to engage the implant interface section 830.

In some embodiments, the housing may have the same diameter along the length. In other embodiments, the housing may include a handle disposed along a portion of the length.

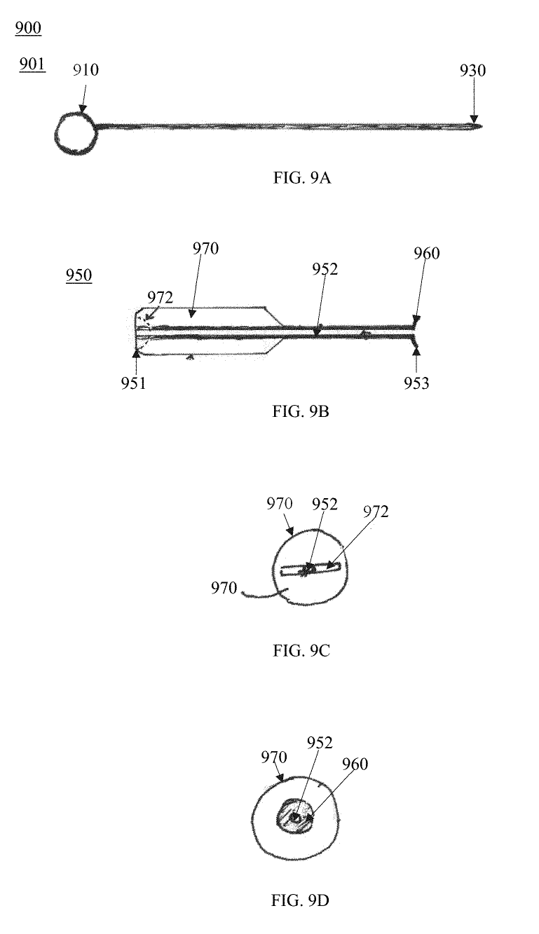

FIGS. 9A-D show another example of an insertion tool 900 according to embodiments. In some embodiments, the tool 900 may include an insert member 900, as shown in FIG. 9A. Similar to the tool 500, the insert member 901 may include a handle 910 disposed at one end and an implant interface section 930 disposed at the opposing end. The implant insertion section 930 may include a pointed or blunt tip onto which an implant may be configured to be disposed.

In some embodiments, the device 900 may include a housing 950 in which and with respect to the insert member 901 may move, as shown in FIGS. 9B-D. By way of example, the housing 950 may include a first end 951, a second end 953, and a length there between. In some embodiments, the housing 950 may include an inner channel 952 that extends along the length. In some embodiments, the housing 950 may include a handle 970 disposed at the first end 951 and extending towards the second 953, a receiving member 960 disposed at the second end 953, and a shaft there between.

In some embodiments, the receiving member 960 may be radially disposed on the shaft so as to surround the inner channel 952. As shown in the FIG. 9B, the receiving member 960 may have a substantially circular shape. In some embodiments, the receiving member 960 may have a convex surface shape as shown in the FIG. 9B. In other embodiments, the receiving member 960 may have a different shape and/or surface shape (e.g., convex, concave, etc.). The shape of the receiving member may correspond to a shape of an implant.

In some embodiments, the handle 970 may include a member 972 disposed at the first end 951 and configured to receive the handle 910 of the insert member 901. In some embodiments, the member 972 may be configured to support the handle 970 during storage and to guide movement of the insert member 901 with respect to the housing 950. For example, the member 972 may be configured to prevent further advancement of the insert member 901 into the inner channel 972 or to prevent rotation of the insert member 901 in respect to the housing 970. The member 972 may have a shape that corresponds to the handle 910. In this example, the member 972 may have a circular depression shape so as to correspond to the circular shape of the handle 910.

In some embodiments, the system may include an insertion guide 1000, for example, as shown in FIG. 10. The insertion guide 1000 may include a channel 1010 in which to guide an insertion tool and a pocket development device. In other embodiments, the insertion tool may act as a guide itself. For example, the housing and/or the insert member may be configured to prevent the further advancement of an implant disposed on the insert member. In some embodiments, the insert member may include an inner channel disposed along the length configured for a guidewire to facilitate positioning of the insert member/implant.

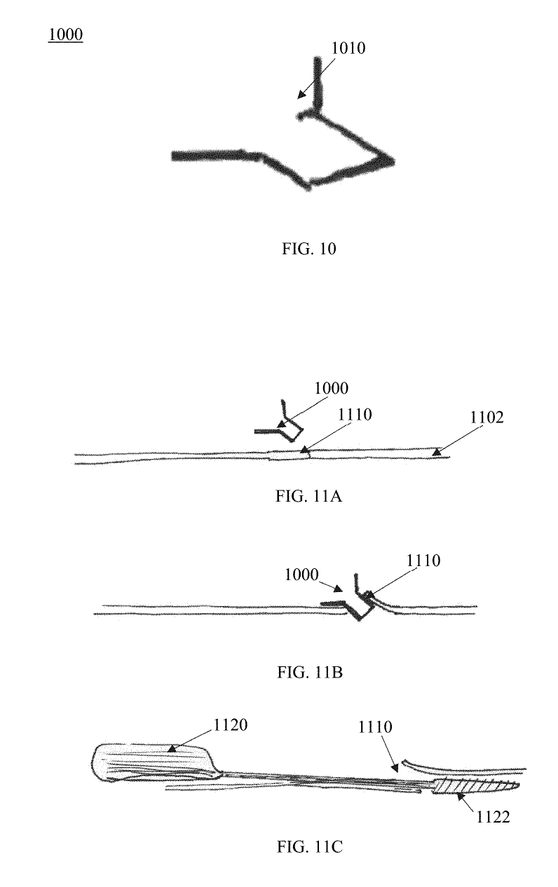

FIGS. 11A-H show an example of method of implanting an implant using the insertion member 900 and the insertion guide 1000, according to some embodiments. In some embodiments, as shown in FIGS. 11A and B, the method may include inserting the insertion guide 1000 in soft tissue 1102 at a soft tissue target site (an access incision) 1110. The method may also omit using the guide 1000.

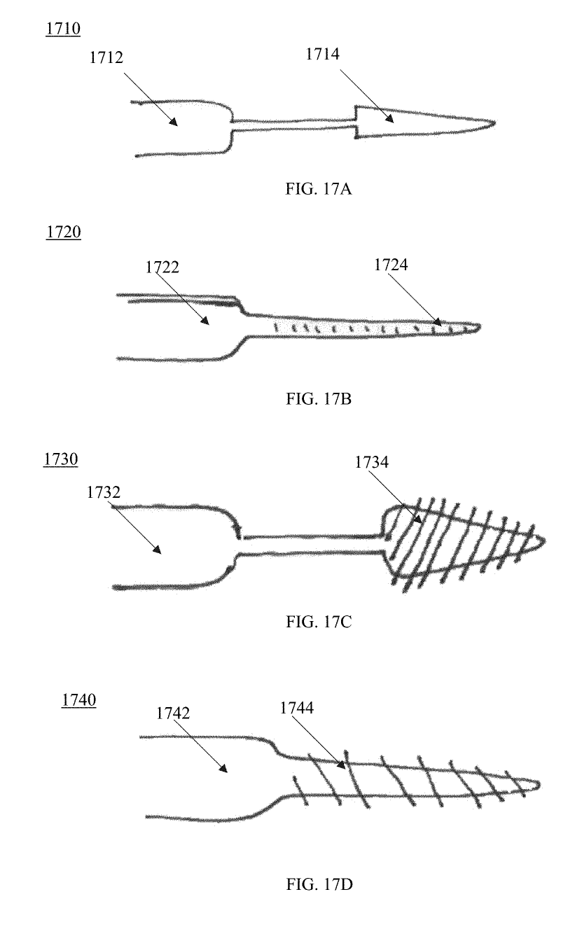

With or without using the insertion guide 1000, a pocket development device 1120 can be inserted into the soft tissue target site or access incision site 1110 to create an access channel in which the insertion tool may be inserted. For example, the pocket development device 1120 may be configured to dissect the soft tissue 1102 to create the channel or pocket 1112 at the access incision site 1110 for an implant, as shown in FIGS. 11C and D. In some embodiments, the pocket development device 1120 may include one or more of slots, grooves, threads, other features, or combination thereof. along its length. In this example, the pocket development device 1120 may include a threaded tip 1122. In some embodiments, the shape of the threaded tip 1122 may correspond to the shape of the implant for optimal placement and thereby reduce migration. As an example, in use, the pocket development device 1120 (e.g., shown in FIG. 11C) can be rotated to advance the device 1120 into the soft tissue 1102 at the access incision site 1110 and to develop the channel 1112 that is configured to receive an implant. The pocket development device 1120 is not limited to the device shown and may be a different pocket development device according to other embodiments. For example, the pocket development device may include any of examples shown in FIGS. 17-20, as well as any other pocket development devices.

Next, as shown in FIG. 11D, the insertion tool 900 on which an implant 1130 is disposed can be inserted into the access channel 1112. The implant 1130 may be casted directly onto the implant interface section 930 of the insertion tool 900 and/or inserted on the implant interface section 930 by the user or in the course of manufacture or assembly.

In some embodiments, to prepare the implant 1130 for insertion into the soft tissue, the implant 1130 may be exposed to an external stimulus (e.g., thermo-manipulation, fluid manipulation, chemical manipulation, etc.) (i) before insertion and/or (ii) before and/or after loading onto the insertion tool 900. In some embodiments, the implant 1130 may be exposed to an external stimulus after insertion into the access channel 1112. For example, the stimulus may include but is not limited to a solution applied to the implant 1130 in vivo and/or or the body's own environment (temperature, solutions, etc.). For example, the implant 1130 may be exposed to activating materials (e.g., applied solutions, bodily fluids, etc.), activating temperature (e.g., heat/room temperature if provided in frozen state), activating energies (e.g., ultrasonic, ultraviolet, laser, radiofrequency, etc.) to cause the implant to change size, shape, firmness, among others, or a combination thereof before and/or after implantation into the soft tissue.

After the insertion tool 900 and/or implant 1130 has been prepared, the insertion tool 900 may be inserted into the pocket 1112 via the access incision 1110, as shown in FIG. 11E. To deliver the implant 1130 and cause the implant 1130 to release from the implant interface section 930, the user can hold the housing 950 via the handle 970 and retract the insert member 901 using the handle 910 with respect to the housing 950 so that the insert member 901 is moved away from the housing 950 and the access channel 1112. Retracting the insert member 901 can cause the implant 1130 to push against the receiving member 960. The receiving member 960 can thereby assist positioning of the implant 1130 during its release from the implant interface section 930 while maintaining appropriate position of the implant 1130 within the access channel 1112. The housing 950 may then be withdrawn from the access incision site 1110 as shown in FIG. 11G, and the incision site 1110 may be closed using sutures, tapes, suture glue or other fastening mechanisms 1170 as shown in FIG. 11H.

In some embodiments, the insertion tool may include other mechanisms configured to move the insert member with respect to the housing and/or release the implant. For example, the housing may include a closed end and the insert member may be configured to advance out of and/or retract into the housing. The housing may include a mechanism that is configured to controllably move the insert member with respect to the housing (e.g., retract and/or advance). The mechanism may include but are not limited to a notch disposed along a channel, threaded/twist release mechanisms, among others, or a combination thereof.

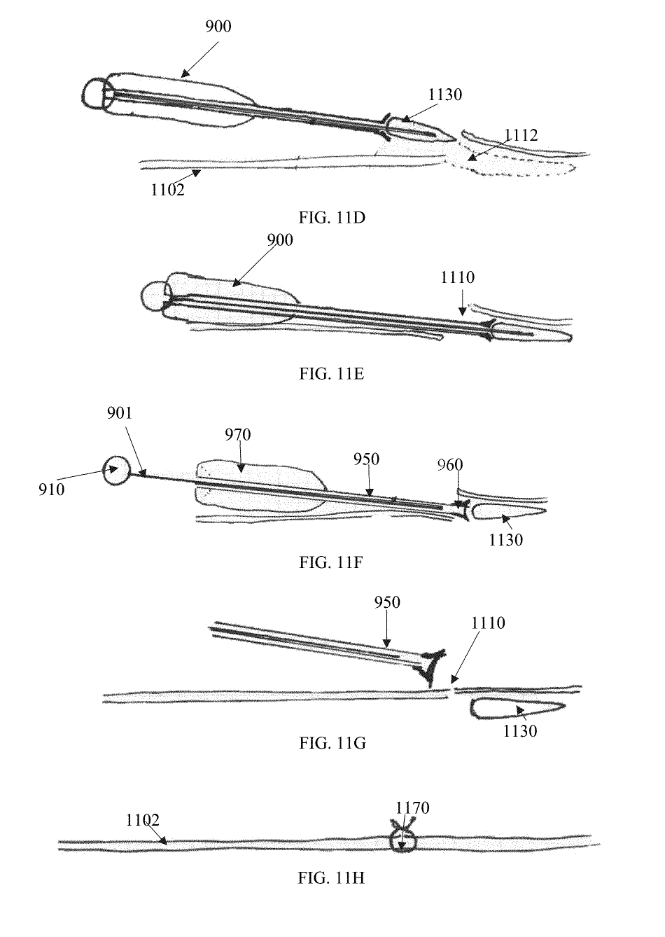

FIG. 12 shows an example of an insertion tool 1200 that includes a mechanism 1280 configured to move an insert member 1220 with the respect to a housing 1250 so that an implant interface section 1230 disposed on the insert member 1220 is advanced parallel to the housing 1250 according to some embodiments. In this example, the insertion tool 1200 may be a pen-like device. In some embodiments, the housing 1250 of the insert tool 1200 may have a first end 1252, a second end 1254, and a length there between. The first end 1252 may be open and the second end 1254 may be closed. In some embodiments, the housing 1250 may include an internal channel corresponding to the opening of the first end 1252 and having a length that is at least a portion of the length of the housing 1250.

In some embodiments, the insert member 1220 that is configured to be disposed within and move along the channel with respect to the housing 1250. The insert member 1220 may include an implant interface section 1230 along a portion of the insert member 1250 that is exposed when the insert member 1220 is advanced with respect to the housing 1250.

In some embodiments, the mechanism 1280 may be disposed along the length of the housing 1250. For example, as shown in FIG. 12, the mechanism 1280 may include a guide channel 1282 and a movable member 1284 that is configured to move along the guide channel 1282. The movable member 1284 may be configured to cause the insert member 1220 to move with respect to the housing 1250. The mechanism 1280 may include a member (not shown) be configured to engage the insert member 1220. For example, the member may be disposed within the channel of the housing 1250 and to which an end of the insert member 1220 may be fixed so that the insert member 1220 moves with respect to the housing when the movable member 1284 moves along the channel 1281. By of example, moving the movable member 1284 towards the first end 1252 causes the insert member 1220 to be advanced with respect to the housing, as shown in FIG. 12. In another example, moving the movable member 1284 towards the second end 1254 causes the insert member 1220 to be retracted with respect to the housing.

In some embodiments, the member 1284 may be configured to removably lock with respect to a position along the channel 1282 to thereby maintain the position of the insert member 1220 with respect to the housing 1250. In some embodiments, the guide channel 1282 may include one or more of grooves, ridges, among others, or a combination thereof, along the length complimentary to the member 1280 and thereby configured to lock the movable member 1284 at one or more positions along the channel 1282.

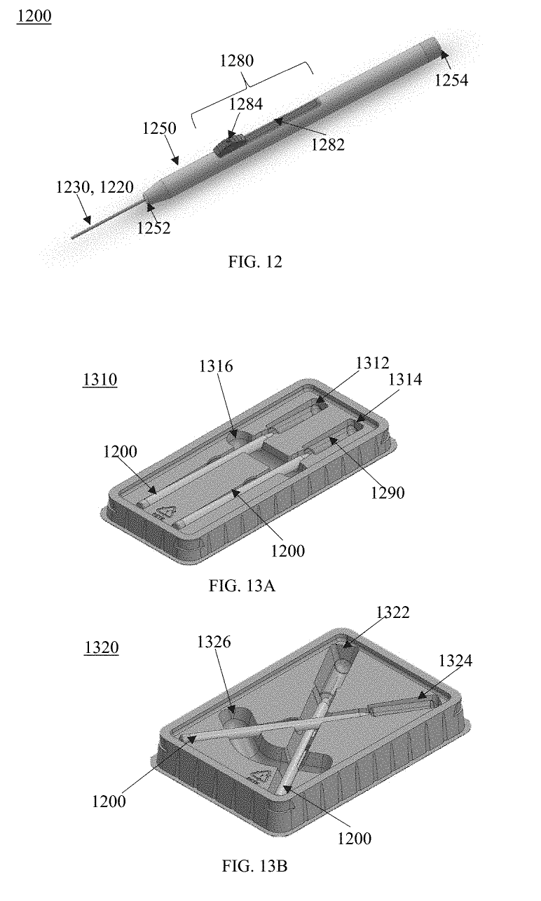

In some embodiments, the insertion tool 1200 may include a cap 1290 that can be removably disposed onto the housing 1250, as shown in FIGS. 13A and 13B. The cap 1290 may be configured to the cover the first end 1252 and the insert member 1220 (if advanced).

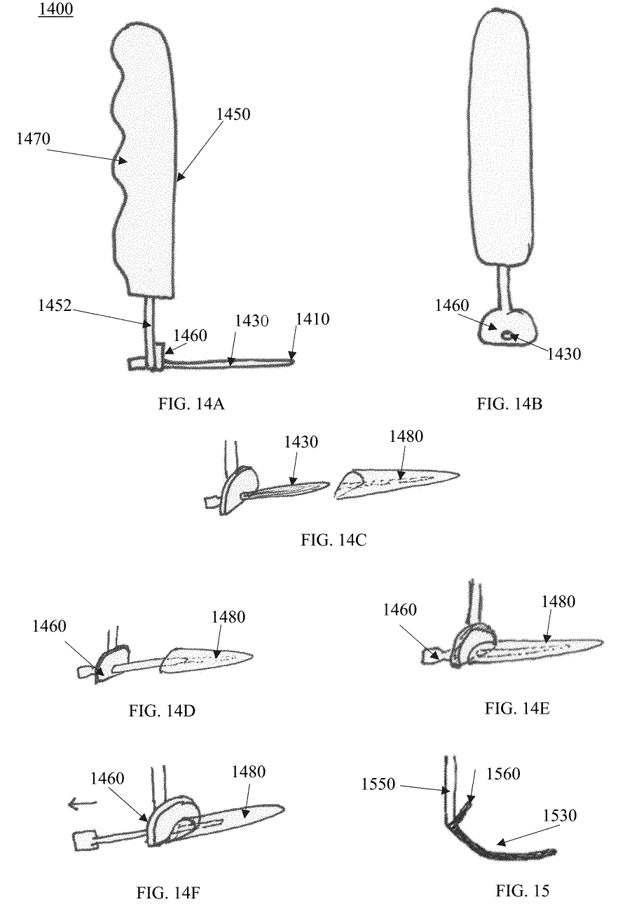

In some embodiments, the insertion tool may include an implant interface section that is disposed on the insert member and not parallel to the housing. In some embodiments, the implant interface section may be perpendicular or offset to the housing. FIGS. 14 and 15 show examples of insertion tools 1400 and 1500 according to some embodiments.

In some embodiments, as shown in FIGS. 14A and B, the insertion tool 1400 may include an insert member 1410. The insert member 1410 may include an implant interface section 1430 disposed at an end. The implant insertion 1430 section may include a pointed or blunt tip onto which the implant may be configured to be disposed. In some embodiments, the insertion tool 1400 may include a housing 1450 in which and with respect to the insert member 1410 may move. By way of example, the housing 1450 may include a first end, a second end, and a length there between. In some embodiments, the housing 1450 may include an inner channel that extends along the length. In some embody embodiments, the tool 1400 may include a lumen 1452 that extends from the housing and corresponding to the inner channel 1452. The inner channel and the lumen 1452 may be configured for a guidewire. In some embodiments, the housing 1450 may include a handle 1470 disposed adjacent to one end, a receiving member 1460 disposed at another end and a shaft there between. As shown in FIGS. 14A and B, the lumen may include an opening on the side of lumen (rather than the end as shown in FIG. 9 of example). The receiving member 1460 may be disposed to surround the lumen opening.

As shown in FIGS. 14A and B, the implant interface section 1430 may be configured to advance in a direction perpendicular to the handle 1470. FIGS. 14C-E show example of loading an implant 1480 onto the implant interface section 1430 so that it abuts against the receiving member 1460. To release the implant 1480 from the implant interface section 1430, the insert member 1410 may be retracted into the receiving member 1460 so that the implant interface section 1430 retracts into the receiving member 1460 and away from the implant 1480 as shown in FIG. 14F.

In some embodiments, the insertion tool may include an implant interface section that may be configured to be disposed at an angle with respect to the housing hen advanced. FIG. 15 shows an example of an implant interface section 1530 that is disposed at a curved angle with respect to a receiving member 1560 and the housing 1550. The angle is not limited to the curvature shown. The implant interface section 1530 when advanced may be configured to be disposed at any angle with respect to the housing, curvature with respect to the housing, among others, or a combination thereof.

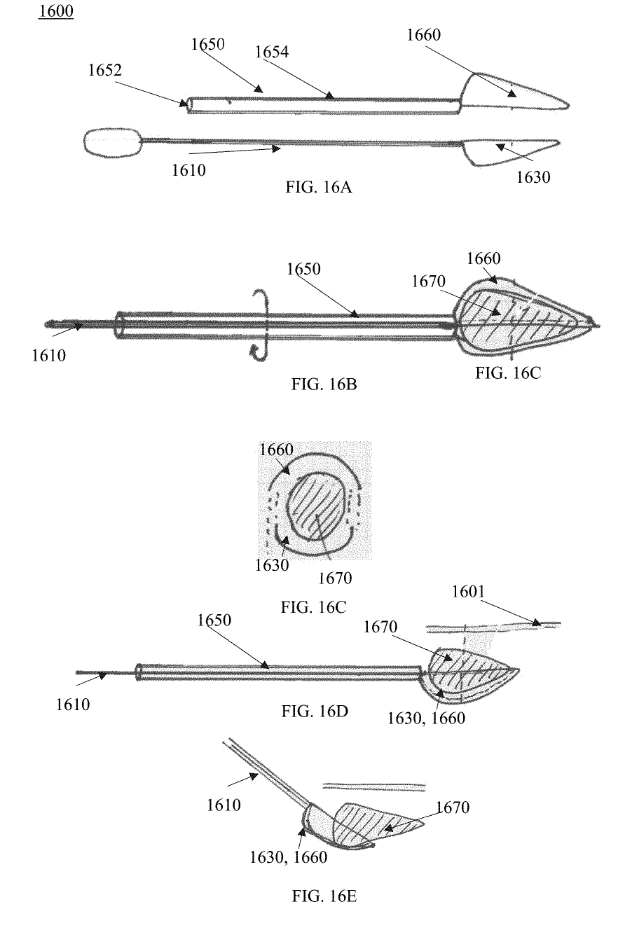

In some embodiments, the insertion tool may include an insert member having an implant interface section in which the implant may be disposed and a housing having a complimentary receiving member than can together form an enclosed area configured to stabilize the implant. FIGS. 16A-E show an example of an insertion tool 1600 according to some embodiments. As shown in FIG. 16A, the insertion tool 1600 may include an insert member 1610 having an implant interface section 1630 disposed at one end. In this example, the implant interface section 1630 may have a curved surface that has a shape that corresponds to the implant to be inserted.

The insertion tool 1600 may also include a housing 1650 that includes an inner channel 1652 disposed along the length, and a receiving member 1660 disposed at an end and configured to surround the opening of the inner channel 1652. In some embodiments, the implant interface section 1630 and the receiving member 1660 may have a complimentary shape. In this example, the members 1630 and 1660 may have a concave shape. As shown in FIGS. 16B and C, when the insert member 1610 is inserted into the housing 1650, the members 1630 and 1660 can form an enclosed area in which an implant 1670 can be positioned.

In some embodiments, the insertion tool 1600 may be inserted into a created pocket 1601 in the closed configuration, shown in FIG. 16B. In some embodiments, to release the implant 1670, the insert member 1610 may be rotated with respect to the housing 1650 to cause the members 1630 and 1660 to overlap, as shown in FIGS. 16D and E. When the members 1630 and 1160 are overlapped, the implant may then be released into the created pocket 1601, as shown in FIG. 16E.

In some embodiments, the system may include a pocket development device configured to create an access channel and/or pocket in the soft tissue in which the implant may be implanted. In some embodiments, the pocket development device may include a handle and one or more dissection sections disposed along the length. In some embodiments, the pocket development device may be solid along its length. In other embodiments, the pocket development device may include a channel disposed along at least a portion of the length.

In some embodiments, the dissection section may be disposed at the end and/or along the length of the pocket development device. In some embodiments, the dissection section may have a fixed shape. For example, the dissection section may have a tapered shape. In other embodiments, the dissection section may have a cylindrical, rounded shape. The dissection section may have a smooth surface. In some embodiments, the dissection section may have a textured surface. In some embodiments, the dissection section may include one or more surface features. By way of example, the surface features may include one or more different surface features. The features may have any number, pattern, size, among others, or a combination thereof. The features may include one or more of threads, notches, grooves, barbs, balls, among others, or any combination thereof. For example, the features may be disposed in a pattern that has a consistent pitch, height, angle, or spacing. In another example, the features may be disposed in a pattern that can vary in pitch, height, angle, or spacing at different points along the length of the pocket development device. In some embodiments, the dissection section may also include one or more openings (e.g., holes, slots, etc.). In some embodiments, the openings may be configured for a suture.

In some embodiments, the dissection section may have a diameter that is smaller and/or larger than a largest diameter of the implant. For example, the dissection section may have a diameter that is larger than the largest diameter of the implant so that the access channel (e.g., pocket) formed in the soft tissue by the pocket development device is larger than the implant. In this example, the pocket development device could be used with an implant that is configured to increase in size to fill the channel when exposed to the corresponding stimulus (e.g., warming temperature, solution, and/or other stimuli). In another example, the dissection section may have a diameter that is smaller than the smallest diameter of the implant, so that the formed access channel can be smaller than the implant to be inserted. In this example, the smaller channel can tamponade any bleeding and/or promote adherence of textured surfaces to the soft tissues.

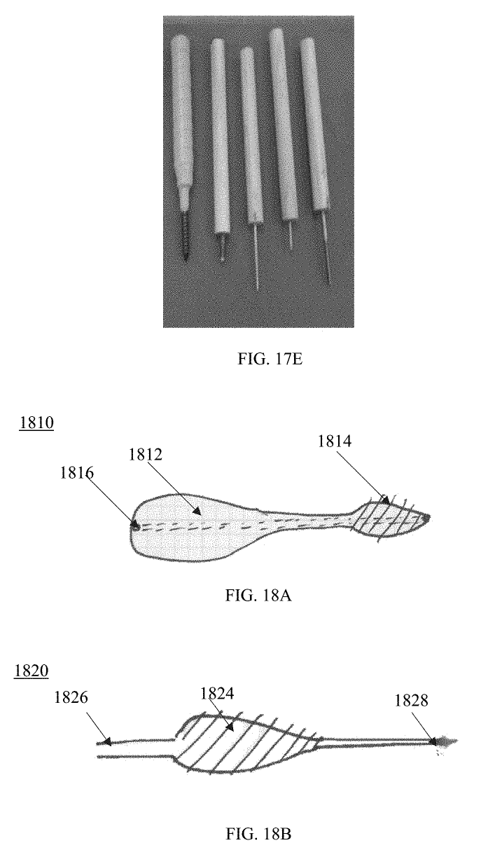

FIGS. 17 and 18A-G show examples of a pocket development device according to some embodiments. In some embodiments, a pocket development device may include a dissection section having any combination of features (e.g., surface feature, dimensions, etc.) shown in these figures, as well as any other features.

As shown in FIG. 17A, the device 1710 may include a handle 1712 and a dissection section 1714. In some embodiments, the dissection section 1714 may be disposed at the tip and extend along a portion of the length of the device 1710. In this example, the dissection section 1714 may have a smooth surface and a tapered shape in which the diameter increases from the tip towards the handle 1710.

FIG. 17B shows a pocket development device 1720 with a different shaped dissection section 1724. In this example, the dissection section 1724 may have a smooth surface. The dissection section 1724 may be disposed at the tip and extend between the tip and a handle 1722.

In some embodiments, the dissection section may have a threaded surface. FIGS. 17C and D show examples of the dissection sections shown in FIGS. 17A and B with threaded surfaces. FIG. 17C shows a device 1730 including a dissection section 1734 that has threaded features along the length and a handle 1732. FIG. 17D shows a device 1740 including a dissection section 1744 that has threaded features along the length of the section and a handle 1742. FIG. 17E shows additional examples of pocket development devices with threaded features.

In some embodiments, the dissection section may include an inner channel and/or be disposed at different positions along the length of the pocket development device. For example, FIG. 18A shows a device 1810 including a handle 1812 and a dissection section 1814 having a threaded surface. In this example, the device 1810 may include a channel 1816 that extends along the length of the device 1810 from the handle 1812 to the tip of the dissection section 1814. This channel 1816 can be configured to accommodate a guide wire, suture, other elements, or a combination thereof to assist in deploying, positioning, or securing the implant. In some embodiments, the device 1810 may include an opening disposed on the handle 1816 and the dissection section 1816 to correspond to the channel.

In some embodiments, the dissection section may be disposed along the length of the pocket development device. For example, as shown in FIG. 18B, the pocket development device 1820 include a dissection section 1824 disposed between the tip 1824 and the handle (not shown). The dissection section 1824 may have one or more threaded features on its surface. In some embodiments, the pocket development device 1820 may optionally include an inner channel 1826 that extends along the length of the device 1820, from the handle to the tip 1828. In other embodiments, the pocket development device 1820 may not include a channel.

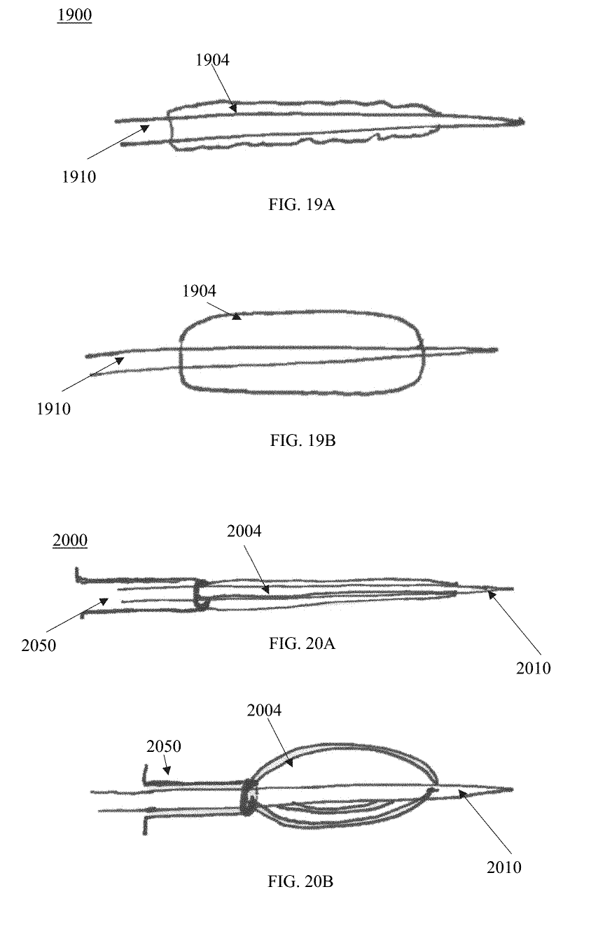

In some embodiments, the dissection section may be configured to expand. For example, FIGS. 19 and 20 show a pocket development device having expandable dissection sections. FIGS. 19A and B show a pocket development device 1900 that includes a shaft 1910 having a length and a pointed tip, and a dissection section 1904 disposed on the shaft. In some embodiments, the dissection section 1904 may be inflatable by a solution and/or gas. FIG. 19A shows the pocket development device 1900 in a deflated state and FIG. 19B shows the dissection section 1904 in an expanded state with respect to the shaft 1910 of the device.

By way of another example, as shown in FIGS. 20A and B, a pocket development device 2000 may include a shaft 2010 having a length and a pointed tip, a housing 2050 that is movable with respect to the shaft 2010 and disposed to surround the shaft 2010, and a dissection section 2004 that includes a plurality of members (e.g., wires) extending from the tip to the housing 2050 of the device. The dissection section 2004 may be attached to the housing 2050 so that when the housing 2010 is advanced with respect to the shaft 2010 towards the tip, the dissection section 2004 can be configured to expand as shown in FIG. 20B from the closed, elongated state shown in FIG. 20A.

In some embodiments, the system may omit the pocket development device, the housing, and/or the insert member. For example, the implant may have a textured outer surface that is configured to create an access channel in the soft tissue like the dissection section. In some embodiments, the pocket development device, the housing, and/or the insert member may be configured to be used with an implant different than those shown and described.

In some embodiments, the system may be part of a kit. In some embodiments, the kit may include one or more of a pocket development device, one or more implant insertion tools, one or more implant, one or more of other procedural materials/instruments (e.g., alcohol pads, betadine pads/swab, lidocaine, needles, suture, tissue glue, implant activating agents, gauze, procedural drape, scalpel. etc.), or any combination thereof. In some embodiments, the implant insertion tool may be preloaded with the implant. By way of example, the kit may include additional implants to be loaded onto the implant insertion tool.

In some embodiments, the kit or package may include a container in the form of an injection molded or vacuum-formed tray, or the like. In some embodiments, the kit or package may include one or more compartments formed in the container. The one or more compartments may be configured to hold one or more insertion tools, one or more implants, one or more pocket development tools, one or more of the other procedural materials/instruments, among other items, or any combination thereof. In some embodiments, the kit or package may include one or more cavities that are each formed in the container and traverses the one or more compartments. The one or more cavities may be configured to provide access to an area below the compartment(s) so as to assist in the release of the system component (e.g., insertion tool) from each compartment.

FIGS. 13A and B show examples of package systems 1310 and 1320, respectively, that are each configured to store and transport one or more the insertion tools 1200 according to some embodiments. In these example, each of the insertion tools 1200 are preloaded with an implant and include a cap 1290 to protect the implant. In some embodiments, the systems 1310 and 1320 may include two compartments that are each configured to hold the insertion tool 1200. In other embodiments, the systems 1310 and 1320 may include more or less compartments that are each configured to hold an insertion tool, one or more compartments that are configured to hold additional tools/devices (e.g., a pocket development tool), one or more implants, one or more of the other procedural materials/instruments, among others, or any combination thereof. In some embodiments, the system may include

In some embodiments, as shown in FIG. 13A, the system 1310 may include compartments 1312 and 1314 that are disposed in parallel. In this example, the tools 1200 may be separately stored. The system 1310 may also include a cavity 1316 that is disposed perpendicular to the compartments 1312 and 1314 so as to traverse the compartments 1312 and 1314.

In some embodiments, as shown in FIG. 13B, the system 1320 may include compartments 1322 and 1324 that are disposed to traverse each other. For example, the compartments 1322 and 1324 may be disposed in an X configuration. In this example, when the tools 1200 are disposed in the compartments 1322 and 1324, the tools 1200 may overlap each other. The system 1310 may also include a cavity 1326 that traverses the compartments 1322 and 1324.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. A soft tissue implant comprising:
a length; and
one or more features disposed on or within the implant configured to engage an implant insertion tool, the one or more features including one or more of an internal channel disposed along the length, a groove, a slot, and/or a notch.

2. The soft tissue implant according to claim 1, further comprising:
one or more regions disposed along the length, the one or more regions having one or more materials, the one or more materials having properties configured to transform from a first configuration to a second configuration based on a stimulus,
wherein the stimulus includes thermo-manipulation, fluid manipulation, chemical manipulation and/or energy manipulation.

3. The soft tissue implant according to claim 2, further comprising:
one or more layers, the one or more layers including one or more of the one or more materials and/or a therapeutic.

4. The soft tissue implant according to claim 3, wherein the one or more materials includes a hydrogel.

5. The soft facial implant according to claim 4, wherein the hydrogel is a polyvinyl alcohol (PVA) hydrogel.

6. A soft tissue implant comprising:
a length;
an inner channel disposed at least partially along the length; and
one or more regions disposed along the length, the one or more regions having one or more materials, the one or more materials having properties configured to transform from a first configuration to a second configuration based on a stimulus,
wherein the stimulus includes thermo-manipulation, fluid manipulation, chemical manipulation, and/or energy manipulation.

7. The soft tissue implant according to claim 6, further comprising:
one or more layers, the one or more layers including one or more of the one or more materials and/or a therapeutic.

8. The soft tissue implant according to claim 7, wherein the one or more materials includes a hydrogel.

9. The soft facial implant according to claim 8, wherein the hydrogel is a polyvinyl alcohol (PVA) hydrogel.

10. The soft tissue implant according to claim 6, further comprising:
one or more features configured to engage an implant insertion tool, the one or more features including one or more of the internal channel, a groove, a slot, and/or a notch.

11. A soft tissue implant system, comprising:
an implant insertion tool, the implant insertion tool including an implant interface section; and
an implant including one or more features configured to engage the implant interface section,
wherein the one or more features of the implant includes one or more of a channel disposed along a length of the implant, a groove, a slot, and/or a notch.

12. The soft tissue implant system according to claim 11, wherein:
the implant includes one or more regions having one or more materials, the one or more materials having properties configured to transform from a first configuration to a second configuration based on a stimulus;
the one or more regions has a smooth surface, a textured surface, and/or a combination thereof and
the stimulus includes thermo-manipulation, fluid manipulation, energy manipulation, chemical manipulation, and/or combination thereof.

13. The soft tissue implant system according to claim 11, wherein the implant is disposed on the implant interface section by being fabricated directly onto the implant interface section.

14. The soft tissue implant system according to claim 11, wherein the implant insertion tool includes a housing, the housing including a channel disposed at least partially along a length of the housing, the channel being in which the implant interface section is movable.

15. The soft tissue implant system according to claim 14, wherein the implant insertion tool includes:
an insert member, the insert member being an elongated member, the insert member including the implant interface section configured to interface with a soft tissue implant; and
a receiving member disposed on the housing and/or the insert member, the receiving member being configured to support and cause release of the implant from the implant interface section.

16. The soft tissue implant system according to claim 15, wherein the receiving member is disposed at an end of the channel of the housing and the receiving member is configured to support and cause the release of the implant from the interface section when the insert member is retracted into the housing.

17. The soft tissue implant system according to claim 11, wherein the implant interface section includes one or more engaging members configured to receive the implant, the one or more engaging members including one or more surface features, the one or more surface features including one or more of a barb, a ball, a groove, and/or a depression.

18. The soft tissue implant system according to claim 11, further comprising:
a dissection section configured to form a soft tissue pocket or channel in soft tissue to receive the implant;
wherein the dissection section has a length that includes one or more surface features disposed in a pattern along the length; and
wherein the one or more surface features includes one or more of a thread, a notch, a groove, a barb, and/or a ball.

20. The soft tissue implant system according to claim 18, wherein the implant and/or the insertion tool includes the dissection section.

21. The soft tissue implant system according to claim 18, further comprising:
a pocket development device, the pocket development device including:
a length; and
the dissection section.
